(19) United States
(12) Patent Application Publication
(10) Pub. No.: US 2010/0189668 A1
(43) Pub. Date: Jul. 29, 2010

(54) Composition for treating herpes simplex viral infections, the method, use and process by which the composition is utilized and the manufacture of the composition

(76) Inventors: Gregory K. McCullough, Primm Springs, TN (US); David S. LeRoy, Nashville, TN (US); James V. Doramus, Nashville, TN (US)

Correspondence Address: Gregory K. McCullough, 7675 Younger Creek Road, Primm Springs, TN, 38476, US

(21) Appl. No.: 12/655,907

(22) Filed: Jan. 11, 2010

Related U.S. Application Data
(60) Provisional application No. 61/205,326, filed on Jan. 21, 2009.

(51) Int. Cl. A61K 36/54; A61K 36/53; A61P 31/22

(52) U.S. Cl. 424/60; 424/739; 424/725

(57) ABSTRACT

This invention relates to a composition, method and process for the treatment of the herpes simplex virus which causes cold sores in humans by a topical application of a blend of essential oils and other natural based materials as a dilutant, all of which may be delivered in either a solid, paste, powder, gel or liquid form to the affected area.

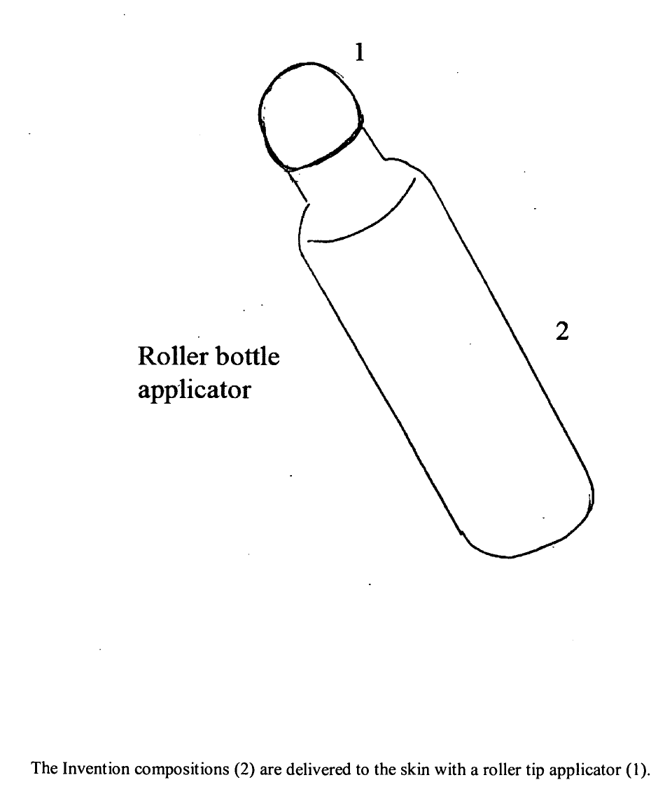

### CROSS REFERENCE AND RELATIONSHIP TO PRIOR APPLICATIONS

This application is related to Provisional Patent Application 61/205,326, filed Jan. 21, 2009.

### STATEMENT REGARDING FEDERALLY SPONSORED RESEARCH

This invention was not created with federally sponsored research and development funds or resources.

### STATEMENT REGARDING SEPARATE LISTINGS, TABLES OR COMPUTER PROGRAMS

This application does not include separately submitted listings, tables or computer programs.

### BACKGROUND OF THE INVENTION

The herpes simplex virus (“HSV”) is implicated as the cause of cold sores and other recurring sores in the human body. Cold sores are usually caused by type I HSV virus, and other sores are usually of the type II virus. Herpes simplex infections are contagious. The virus is spread from person to person by kissing or other close contact with sores or even from contact with apparently normal skin that is shedding the virus. People are most contagious when they have active blister-like sores. Once the blisters have dried and crusted over (within a few days), the risk of contagion is significantly lessened. However, a person infected with HSV can pass it on to another person even when a sore is not present. The HSV enters the body after contact with an infected host. Following an incubation period of four to ten days the infection enters a prodomal stage, then progresses to skin eruptions which manifest as ulcerated lesions. These ulcers gradually coalesce, crust over, heal and then reenter the latency period.

After the first infection, the virus enters the nerve cells and travels up the nerve until it comes to a place called a ganglion. There, it lays quietly in a stage that is called a “dormant” or “latent” period. At times, the virus can start multiplying again and travel down the nerve to the skin, causing new cold sores. Often this process is known to the person because of a tingling sensating and the appearance of a slight bump on the skin. The triggering or burning events are not clearly understood, but it is known that some conditions seem to be associated with recurrences, including
fever, colds, or the flu
ultraviolet radiation (exposure to the sun),
stress,
changes in the immune system,
hormonal changes, such as menstruation, and
trauma to the skin.

Sores have a tendency to recur in more or less the same place. Such recurrences may happen often (for example, once a month) or only occasionally (for example, once or twice a year). Sometimes there is no apparent cause of the recurrence.

The impact of HSV has devastating social and psychological consequences, as well as additional health risks, including the suggestion that HSV may be a cause of blindness, increased risk of cervical cancer and other risks, especially in individuals with compromised immune systems, as in those individuals who have Auto Immune Deficiency Syndrome (AIDS).

Although various treatments have been devised they are often not efficacious. Among these treatments are acyclovir, povidone-iodine, idoxuridene, or trifluorothyidine. Acyclovir is reported to inhibit the activity of several herpes viruses. However, there is a evidence that herpes viruses, especially in individuals with AIDS, have mutated and acquired a resistance to acyclovir.

The common method of administration of acylclovir is orally, and transdermal administration of the compositions has not been effective. Many of the topically administered compositions are oil-based, and the oil is not only messy but actually inhibits the absorption of the compositions into the skin and the site where the virus is resident. Other remedies have tried to incorporate various other compositions which alleviate itching and promote healing of the lesions, but they do not successfully attack the virus at its source.

Johnson in U.S. Pat. No. 6,211,243 describes the treatment of cold sores with benzalkonium chloride, using an applicator for applying the compostions directly to the infected site. His disclosures are incorporated herein by reference.

As disclosed in Johnson, examples of conventional application methods and compositions are provided in WO 98/42188 and in WO 98/11778 by Squires, both of which are hereby incorporated by reference. In WO 98/42188 at page 9, lines 12-18 and in WO 98/11778 at page 5, lines 22-30, it is stated that the composition is applied by “spraying, dabbing, dusting, swabbing, sponging, brushing, pouring, dispensing, covering or heavily coating.” The stated objective of these techniques for applying the composition is to insure that the composition remains on the infected area. Like other conventional treatment compositions, the composition has a viscosity and/or tackiness which enables it to remain on the surface of the infected area. A portion of such compositions may eventually penetrate beyond the surface of the disordered tissue such as the outer surface of skin or a lip, however, the viscosity of the composition combined with the application technique prevents such compositions from achieving effective penetration.

Another example of conventional application methods and compositions is provided in U.S. Pat. No. 5,753,270 issued to Beauchamp et al., which is also incorporated by reference. U.S. Pat. No. 5,732,270 discloses a composition which includes: (a) an antiseptic and/or anesthetic compound which is (i) a terpene such as menthol or eucalyptol, (ii) a phenolic compound such as thymol, or (iii) an alcohol such as menthol; (b) a quaternary ammonium antiseptic compound such as benzethonium chloride; and (c) an antiseptic compound containing iodine, salts thereof and/or complexes thereof dissolved in an organic skin penetrating solvent such as a mixture of water and acetone. The methodology is described in the examples provided at columns 5-7 as involving the liberal application of the composition to the afflicted area in a sequence such as 3 to 4 applications over a one minute period which is then repeated every 3 minutes over a 10 minute period. The entire procedure is then repeated after approximately ½ to 1 hour for 2 to 3 hours or until activity is stopped in healing is evident. The use of a cotton swab is mentioned in this reference and is shown at column 6, lines 10-11 for applying the composition.

Although it is mentioned in U.S. Pat. No. 5,753,270 at column 3, lines 44-49 that the formulations may be prepared as a gel, cream, a lotion, an ointment, or a paste the preferred embodiment appears to be a solution having an aqueous solvent system. It is noted at column 3, lines 6-9 that although use of water and acetone as a solvent is preferred such a solvent is not considered essential to the overall synergistic action of the formulation. In any event, the formulation appears not to rely on either its viscosity or tackiness to ensure that the formation is maintained on the surface of the afflicted area as do most conventional treatments. The methodology involves the very frequent reapplication of the formulation to the afflicted area. Some of the formulation may be absorbed into the skin, however, a significant portion is likely rapidly evaporated, and therefore not as effective, due to the high content of water and acetone.

The active agents disclosed in Beauchamp U.S. Pat. No. 5,753,270, which is incorporated herein by reference and which are discussed above include at least one compound which is an antiseptic and/or an anesthetic. The primary examples of such compounds: menthol, eucalyptol, and thymol are either obtained from natural sources such as naturally occurring oils or are derived from such oils. Eucalyptol is described as being an essential oil and a terpene ether. Thymol is derived from thyme oil or other oils. Menthol is obtained from peppermint oil or other oils. Other suitable compounds are also recited in the claims as including: eugenol, camphor, hexetidine or anethol. While the basis for inclusion of hexetidine in this grouping is not clear, the other chemicals are also obtained from natural sources or are derived therefrom. Eugenol is obtained by extraction of clove oil and then chemical modification. Camphor is a ketone which occurs naturally in the wood of the camphor tree. Anethol is derived from anise or fennel oils. As noted, the compounds disclosed in Beauchamp may be useful, particularly as antifungal agents; the efficacy is not clearly delineated. Further Beauchamp appears to take a “shotgun” approach to a remedy by bundling a combination of compositions, some of which may not be efficacious or may interfere with the action of the other ingredients.

### BRIEF SUMMARY OF THE INVENTION

The present invention relates to the treatment of herpes simplex viruses in humans, and in particular those viruses associated with the common malady known as cold sores. The inventive composition and method combines a certain combination of essential oils and a transport composition to assist in the absorption of the oils by the skin, to deliver the agents to the site of the herpes virus. The preferred embodiment is a combination of essential oils such as rosemary, wintergreen, cinnamon, 2-phenethyl propionate and thyme oil, diluted to a concentration of less than fifteen percent in a solution of ethyl lactate.

### BRIEF DESCRIPTION OF THE DRAWINGS

The Drawing submitted with this application discloses a device for applying the subject of this invention directly to the skin. A bottle (1) with a roller cap (2) is filled with the composition described herein and the composition is applied directly to the affected area

### DETAILED DESCRIPTION OF THE INVENTION

This invention relates to a composition for the treatment of HSV, the manufacture of that composition and the method, process and use of the composition (collectively, the “Invention”). The Invention involves a topical application of a blend of essential oils and other natural based materials as a dilutant, all of which may be delivered in either a solid, paste, powder, gel or liquid form to the affected area.

Although the Invention may be delivered in a variety of forms and compositions of essential oils, the preferred embodiment, by way of explanation but limitation, is a liquid dispensed in a roller tip bottle comprised of a blend of the following materials:
Ethyl Lactate (corn ester) or other medium for dissolving and delivering the essential oils,
Rosemary Oil,
Wintergreen Oil,
2-phenethyl propionate (derived from peanuts but without the amino acids that cause reactions in people who are allergic to peanuts and peanut products),
Cinnamon Oil
Thyme Oil.

Ethyl lactate, also known as lactic acid ethyl ester, is a monobasic ester formed from lactic acid and ethanol, commonly used as a solvent. It is a “green” composition and easily biodegradable. Ethyl lactate is found naturally in small quantities in a wide variety of foods and the odor of ethyl lactate when dilute is mild, buttery and creamy with hints of fruit and coconut. Its low toxicity makes it an ideal carrier for the essential oils in this invention and its solvent characteristics permit it to easily penetrate the skin and deliver the oils to the locus of the HSV.

As disclosed in Tanaka et al, U.S. Pat. No. 6,703,053 there are even instances where natural essential oils may exhibit strong powerful results against the HSV virus on contact. The active ingredients vary from Terpenes, Sesquiterpenes through to Ketones, Aldehydes, Esters, to Alcohols, and phenols. Generally, the essential oils are the refined active ingredients from flowers, leaves and roots of plants including lavender, basil, calendula, lemon, tee tree, cinnamon, citronella, clove, eucalyptus, geranium, niqouli, oregano, peppermint, thyme, ginger, patchouli, cedar wood, Melissa, cajeput, rosemary and many others.

However, the use of essential oils is by itself an art of blending and selecting relative proportions which would not be obvious to one of ordinary skill in the art, and the delivery of the composition to the HSV is a problem heretofore unsolved. By the nature of an essential oil the dilution and delivery of the composition and the utilization of materials which are not in themselves harmful is not obvious. In this invention the exact blend of the oils and other materials may vary, but the essential oils as a whole are generally less than fifteen percent of the solution, and are carefully blended in a proportion which maximizes the benefit of the application of these materials. These materials act synergistically to disrupt HSV and prevent it from entering into the eruptive stages, or if erupted, accelerate the healing cycle and promote relief from the infection. The synergism of the compositions described above are dependent upon their relevant proportions in the composition and it has been demonstrated through clinical evidence that the most efficacious combination is comprised of 3-7.5% rosemary oil, 0.5-2.5% wintergreen oil, 0.5-1.5% cinnamon oil, 1-3% 2-phenethyl propionate and 0.25-75% thyme oil in a solution of ethyl lactate. Notwithstanding the relative proportions of the essential oils, the preferred embodiment utilizes a 85-90% solution of ethyl lactate. The advance of this invention over the prior art is in the particular selection and combination of the essential oils and the use of ethyl lactate as a dispensing and delivery medium.

The invention as described herein differs from the prior art in both the selection of the pharmacological components and the mechanism for the delivery of the components. Prior embodiments rely on materials which have an excessive drying action, such as Beauchamp, which endorses, for example, compositions of acetone and water, and provide general guidelines for materials which purportedly counteract HSV. However, for skin already damaged by HSV excess drying promoted by acetone can prolong the problem rather than promote healing. Furthermore, the combination of essential oils is critical. Because of their different characteristics the careful selection of relative strengths is essential to efficacy, and this invention discloses a combination which carefully selects and blends the essential oils in a formula which is not otherwise known or disclosed. The combination of essential oils disclosed herein permits the efficacy of each to be available to counteract HSV, while at the same time not being so strong or dominant as to overshadow or cancel the effect of the other ingredients.

In particular, the embodiment described herein does not use the acetone-water or aqueous solvent systems proposed in Beauchamp, and the combination of the essential oils described herein with the ethyl lactate acts synergistically and gives an unexpected and innovative remedial effect. Combinations of these materials into a cold sore formulation have not heretofore been disclosed.

In addition to these compositions the following compositions may be added to the embodiment:
(a) Allantoin, 0.5 to 2 percent.
(b) Aluminum hydroxide gel, 0.15 to 5 percent.
(c) Calamine, 1 to 25 percent.
(d) Cocoa butter, 50 to 100 percent.
(e) Cod liver oil, 5 to 13.56 percent, in accordance with §347.20(a)(1) or (a)(2), provided the product is labeled so that the quantity used in a 24-hour period does not exceed 10,000 U.S.P. Units vitamin A and 400 U.S.P. Units cholecalciferol.
(f) Colloidal oatmeal, 0.007 percent minimum; 0.003 percent minimum in combination with mineral oil in accordance with §347.20(a)(4).
(g) Dimethicone, 1 to 30 percent.
(h) Glycerin, 20 to 45 percent.
(i) Hard fat, 50 to 100 percent.
(j) Kaolin, 4 to 20 percent.
(k) Lanolin, 12.5 to 50 percent.
(l) Mineral oil, 50 to 100 percent; 30 to 35 percent in combination with colloidal oatmeal in accordance with §347.20(a)(4).
(m) Petrolatum, 30 to 100 percent.
(n) [Reserved]
(o) Sodium bicarbonate.
(p) [Reserved]
(q) Topical starch, 10 to 98 percent.
(r) White petrolatum, 30 to 100 percent.
(s) Zinc acetate, 0.1 to 2 percent.
(t) Zinc carbonate, 0.2 to 2 percent.
(u) Zinc oxide, 1 to 25 percent.

In addition to these materials a sunscreen material may be added to the Invention to reduce the effect of ultraviolet exposure as a triggering mechanism.

The preferred embodiment of this invention is a composition which is manufactured by combining the essential oils of wintergreen, rosemary, thyme, 2-phenethyl propionate and cinnamon in the proportions as described herein with ethyl lactate. To insure the miscibility of the composition with some embodiments it is necessary to use ultra high speed mixing devices as a precursor to packaging the composition in the applicator bottles. Furthermore, when the materials are used in plastic containers the containers should be fluorinated to prevent degradation of the materials over time.

At the first sign or indication that the virus has become active an individual begins applying the Invention to the affected site. The Drawing shows a typical delivery vehicle such as a roller tip bottle for transdermal application. Although a variety of healing mechanisms may be responsible for the improvement caused by the Invention and are included within this Invention, one particular mechanism contemplated by the Invention is the disruption of the viral replication cycle by interfering with the proliferation of the virus as it gets closer to the surface of the skin, as by dissolving part of the virus shell. By stopping or inhibiting the viral replication the individual's natural immune responses repair the affected cells. If the virus does result in a skin outbreak the Invention reduces the healing time by both inhibiting the viral replication and by the beneficial skin effects of the essential oils.

Of particular importance is that current anti-viral compounds are reportedly losing their efficacy as the HSV virus develops mutated resistance to the traditional compounds. The Invention presents an agent that is expected to interrupt the development of even these resistant strains.

The use of the composition described is easily demonstrated in clinical examples as set forth below.

### Example 1

### Treatment Protocols

A typical synergistic combination of the medicinal agents is as follows: 5% rosemary oil, 1.5% wintergreen oil, 1% thyme oil, 2% cinnamon oil, 2% 2-phenethyl propionate, 0.5% thyme oil and 88% ethyl lactate. The present invention has been tested on volunteer patients. A full scale clinical study has not been undertaken.

Directions to the patients are as follows:
1. At the first sign of a tingling sensation in the area usually afflicted by HSV the patient should begin the treatment.
2. The composition should be liberally applied to the locus of the tingling sensation. The initial application should insure that the affected area is “wetted” with the composition to allow it to penetrate the skin.
3. After the initial application, the process is repeated at intervals of one to two hours, or until the patient is no longer aware of the tingling sensation.
4. At such time as the symptoms appear to be alleviated the patient then decreases the frequency of application and applies the composition every eight hours.
5. After three days if the affected area has not erupted into a blister the treatment may be discontinued until the next outbreak is signaled by the itching sensation.

When this regimen was applied by patients in the manner set forth herein excellent results were obtained, and the patients reported immediate healing and alleviation of the symptoms of HSV. Patients reported that the procedure had been extremely beneficial and they were relieved of the pain and embarrassment of open cold sore lesions.

If the treatment is initiated after a blister has erupted a slightly different treatment protocol is suggested. The patient begins by gently applying the composition to the blister, and may experience a brief stinging sensation. After approximately five minutes the affected area is treated again, and then repeated in five minute intervals for approximately thirty minutes. At the end of this time the treatment is then repeated at fifteen minute intervals for a period of two to three hours, and then again hourly while the patient is awake. On the second day of treatment the patient may then begin to find that the lesion is crusting or hardening. The composition is then applied to the lesion every two to three hours, to keep it softened and facilitate healing. By following this process patients were able to expedite the healing of their lesions, and reported that the lesions were dramatically healed, often in the one to two day range instead of the usual and expected period of seven to ten days.

### Clinical Example 1

A middle aged female reported a long and painful history of cold sores which manifested large and unsightly lesions. When the patient first obtained the composition described in this invention a lesion had already erupted. She followed the protocol set forth above. Within one day the sore had begun to coalesce. It appeared to the patient that the composition halted the progress of the sore beginning with the first application and after two days the scab sloughed off. She reported that this was an exceptional and unexpected result. Within a week the sore was completely healed and returned to normal.

### Clinical Example 2

A middle aged female was given the composition in anticipation of potential HSV outbreaks, which had a history of occurring. At the first sign of a burning or tingling sensation she began to follow the protocol described above. She reported that the sensation she experienced was the precursor to an outbreak, but in this instance the immediate application of the composition halted the progress of the outbreak. The tingling sensation diminished, then halted and the cold sore never broke through the skin. She reported no further symptoms.

### Clinical Example 3

An eighty year retired physician was given the composition for the purpose of evaluating its clinical efficacy. He reported that he had a potential outbreak of HSV and that he applied the composition to the area which was then beginning to burn and tingle. He reported that it caused the burning and tingling to stop, and that the HSV did not result in a lesion.

### Clinical Example 4

A middle aged female with a history of breast cancer reported that she had an outbreak of shingles, a type of herpes virus that causes chicken pox, on her face and her nose, including inside of her nose. For over one year she had been subjected to constant pain and frustration. She applied the composition to the affected areas once a day for less than a week and reported that the lesions disappeared and she was symptom free and pain free.

### Clinical Example 5

A young adult female had an external lesion caused by HSV and believed that another was in the process of erupting because of the burning and tingling sensation on the side of her face. She applied the composition hourly for eight hours and began to see significant improvement within several hours. After the eight hours the existing lesion was significantly healed, and the other potential lesion never erupted.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the Invention should be accorded the broadest interpretation so as to encompass all such modifications.

The embodiments of the invention in which an exclusive property or privilege is claimed are as follow:

1. A composition for the treatment HSV, the preparation comprising a mixture of
(a) a synergistic blend of essential oils, and
(b) a carrier material selected for its compatibility with human skin and also capable of delivering the essential oils described above both transdermally and subcutaneously.

2. The preparation of claim 1 above wherein one of the essential oils is rosemary.

3. The preparation of claim 1 above wherein one of the essential oils is thyme.

4. The preparation of claim 1 above wherein one of the essential oils cinnamon.

5. The preparation of claim 1 above wherein one of the essential oils is wintergreen.

6. The preparation of claim 1 above wherein one of the essential oils is rosemary, which oil is then added to and mixed with one or more of the essential oils of cinnamon, thyme and wintergreen and 2-phenethyl propionate.

7. The preparation of claim 1 above wherein the carrier material is ethyl lactate.

8. The preparation of claim 6 above wherein a sunscreen material is added to the composition.

9. The preparation of claim 6 wherein one or more of the following materials are added to the composition:
(a) Allantoin, 0.5 to 2 percent.
(b) Aluminum hydroxide gel, 0.15 to 5 percent.
(c) Calamine, 1 to 25 percent.
(d) Cocoa butter, 50 to 100 percent.
(e) Cod liver oil, 5 to 13.56 percent, in accordance with §347.20(a)(1) or (a)(2), provided the product is labeled so that the quantity used in a 24-hour period does not exceed 10,000 U.S.P. Units vitamin A and 400 U.S.P. Units cholecalciferol.
(f) Colloidal oatmeal, 0.007 percent minimum; 0.003 percent minimum in combination with mineral oil in accordance with §347.20(a)(4).
(g) Dimethicone, 1 to 30 percent.
(h) Glycerin, 20 to 45 percent.
(i) Hard fat, 50 to 100 percent.
(j) Kaolin, 4 to 20 percent.
(k) Lanolin, 12.5 to 50 percent.
(l) Mineral oil, 50 to 100 percent; 30 to 35 percent in combination with colloidal oatmeal in accordance with §347.20(a)(4).
(m) Petrolatum, 30 to 100 percent.
(n) [Reserved]
(o) Sodium bicarbonate.
(p) [Reserved]
(q) Topical starch, 10 to 98 percent.
(r) White petrolatum, 30 to 100 percent.
(s) Zinc acetate, 0.1 to 2 percent.
(t) Zinc carbonate, 0.2 to 2 percent.
(u) Zinc oxide, 1 to 25 percent.

10. A method of treating HSV comprising the application of a mixture of the composition described below to the affected area of skin on the human body which is susceptible to HSV:
(a) a synergistic blend of essential oils, and
(b) a carrier material selected for its compatibility with human skin and also capable of delivering the essential oils described above transdermally.

11. A method of treating HSV comprising the application of a mixture of the composition described in claim 10 to the affected area of skin on the human body which is susceptible to HSV wherein one of the essential oils is rosemary.

12. A method of treating HSV comprising the application of a mixture of the composition described in claim 10 to the affected area of skin on the human body which is susceptible to HSV wherein one of the essential oils thyme.

13. A method of treating HSV comprising the application of a mixture of the composition described in claim 10 to the affected area of skin on the human body which is susceptible to HSV wherein one of the essential oils is cinnamon.

14. A method of treating HSV comprising the application of a mixture of the composition described in claim 10 to the affected area of skin on the human body which is susceptible to HSV wherein one of the essential oils is wintergreen.

15. A method of treating HSV comprising the application of a mixture of the composition described in claim 10 to the affected area of skin on the human body which is susceptible to HSV wherein one of the essential oils is 2-phenethyl propionate

16. A method of treating HSV comprising the application of a mixture of the composition described in claim 10 to the affected area of skin on the human body which is susceptible to HSV wherein one of the essential oils wherein one of the essential oils is rosemary, which oil is then added to and mixed with one or more of the essential oils of cinnamon, thyme, 2-phenethyl propionate and wintergreen.

17. A method of treating HSV comprising the application of a mixture of the composition described in claim 6 to the affected area of skin on the human body which is susceptible to HSV wherein one or more of the following materials are added to the composition:
(a) Allantoin, 0.5 to 2 percent.
(b) Aluminum hydroxide gel, 0.15 to 5 percent.
(c) Calamine, 1 to 25 percent.
(d) Cocoa butter, 50 to 100 percent.
(e) Cod liver oil, 5 to 13.56 percent, in accordance with §347.20(a)(1) or (a)(2), provided the product is labeled so that the quantity used in a 24-hour period does not exceed 10,000 U.S.P. Units vitamin A and 400 U.S.P. Units cholecalciferol.
(f) Colloidal oatmeal, 0.007 percent minimum; 0.003 percent minimum in combination with mineral oil in accordance with §347.20(a)(4).
(g) Dimethicone, 1 to 30 percent.
(h) Glycerin, 20 to 45 percent.
(i) Hard fat, 50 to 100 percent.
(j) Kaolin, 4 to 20 percent.
(k) Lanolin, 12.5 to 50 percent.
(l) Mineral oil, 50 to 100 percent; 30 to 35 percent in combination with colloidal oatmeal in accordance with §347.20(a)(4).
(m) Petrolatum, 30 to 100 percent.
(n) [Reserved]
(o) Sodium bicarbonate.
(p) [Reserved]
(q) Topical starch, 10 to 98 percent.
(r) White petrolatum, 30 to 100 percent.
(s) Zinc acetate, 0.1 to 2 percent.
(t) Zinc carbonate, 0.2 to 2 percent.
(u) Zinc oxide, 1 to 25 percent.
